(19) United States
(12) Patent Application Publication
(10) Pub. No.: US 2021/0222105 A1
(43) Pub. Date: Jul. 22, 2021

(54) A Microfluidic Cartridge, A Kit And An Assay

(71) Applicant: CENTRE FOR CELLULAR AND MOLECULAR PLATFORMS, Bangalore (IN)

(72) Inventors: Taslimarif SAIYED, Bangalore (IN); Satish KALME, Bangalore (IN); Pooja MEHTA, Bangalore (IN)

(73) Assignee: CENTRE FOR CELLULAR AND MOLECULAR PLATFORMS, Bangalore (IN)

(21) Appl. No.: 17/058,872

(22) PCT Filed: May 27, 2019
(86) PCT No.: PCT/IB2019/054386

§ 371 Date: Nov. 25, 2020

(30) Foreign Application Priority Data
May 26, 2018 (IN) 201841011109

(51) Int. Cl. C12M 3/06; C12M 3/00; C12M 1/34; C12Q 1/18

(52) U.S. Cl. CPC C12M 23/16; C12Q 1/18; C12M 41/46; C12M 23/42

(57) ABSTRACT

The present disclosure relates to microfluidic cartridge for determining resistance or susceptibility of microorganism towards agent selected from a group comprising antibiotics, antiviral, antifungal, pesticide and insecticide. The disclosure further relates to a kit comprising microfluidic cartridge containing components selected from a group comprising growth media, agent, reagent, sensor and any combination thereof. The microfluidic cartridge and the kit described herein enables rapid and personalized determination of susceptibility or resistance of microorganisms towards agent. The disclosure also relates to an assay for determining the susceptibility or resistance of microorganisms towards agent employing the said microfluidic cartridge.

### TECHNICAL FIELD

The present disclosure relates to microfluidic cartridge for determining resistance or susceptibility of microorganisms towards agents selected from a group comprising antibiotic, antifungal. The present disclosure further relates to a kit comprising microfluidic cartridge containing components selected from a group comprising growth media, agent, reagent, sensor and any combination thereof. The present disclosure further relates to an assay for determining the susceptibility or resistance of microorganisms towards anti-microbial selected from a group comprising antibiotic, antifungal. The present disclosure also relates to use of the microfluidic cartridge in screening agent to assess their response against microorganisms.

### BACKGROUND OF THE DISCLOSURE

The role of molecular diagnostics is critical in today's global health care environment. In the developing world, 95% of deaths are due to lack of accurate diagnostics and the associated follow-on treatment of infectious diseases. Factors, such as rapid pathogen mutation rates, transformation of nonhuman pathogen with human pathogens have added to the challenge of managing novel infectious diseases. Current laboratory culture methods necessary to detect infectious pathogens take a day or more to provide results.

Diagnostic testing for certain types of infections needs to be repeated periodically to measure response to therapy and monitor the disease condition. These issues highlight the need for rapid, portable diagnostic point-of-care (POC) devices at ports of entry to prevent global spread of infections. However, to date, only very limited portable in vitro diagnostic devices have been successfully developed and commercialized. Yet, there are several significant disadvantages in the available portable in vitro devices, such as need to run external positive and negative assay controls. When the positive and negative assay controls are not part of the test device, inherent lot-to-lot reagent variations and/or degradation can compromise validation of assay results. For the end-user, there is an added cost and time required to acquire additional control reagents and devices and perform the separate control assays. Moreover, it is often left up to the end-user to determine the appropriate frequency of control testing, which places an additional burden on the user. These disadvantages become increasingly significant when multiplex assays are run and are of particular concern in resource-poor settings.

An additional challenge is portability. Although the benefits of the use of fluorophores as probes for in-vitro diagnostic assays are well known. However, the most commonly available forms of equipment for assay are large, complex to use, relatively slow and rely on expensive confocal optics. These attributes make such equipment unsuitable for fully integrated sample to solution in vitro testing in remote locales and on-site at the point of care, where such equipment is required to be rugged, fast, compact, inexpensive and easy to use. Moreover, the existing devices requires active flow controls like gates, valves and mixing ports in the device channels. These requirements increase the cost and complexity of the cartridge. Also, the currently available device does not have multiplexing capacity in terms of testing various antibiotics and variable concentrations of the various antibiotics.

The present disclosure is directed to overcome one or more problems stated above or any other limitations associated with the prior arts.

### SUMMARY OF THE DISCLOSURE

The present disclosure addresses the problem of reliable and valid detection of optical read-outs in a microfluidic cartridge by providing several improved features and integrating both process controls and positive and negative assay controls into the cartridge design. The microfluidic cartridges of the present disclosure offer several considerable advantages to the end-user.

The microfluidic cartridge of the present invention comprises—
a non-reactive base comprising a plurality of inlaid, spatially isolated arrays
each array comprising at least one reaction chamber wherein said reaction chamber is in fluid communication with an inlet port and an outlet port opposed to the inlet port, said base having at least one microchannel in fluid communication with all the inlet ports,
at least one separate microchannel in fluid communication with all outlet ports
and a laminar film disposed over the arrays and reaction chambers so as to sealably isolate their contents from the external environment.

The present disclosure further relates to a kit comprising the microfluidic cartridge containing components selected from a group comprising growth media, reagent, sensor and any combination thereof.

The present disclosure further relates to an assay for determining resistance or susceptibility of microorganisms towards agents selected from a group comprising antibiotic, antifungal, antiviral, pesticide and insecticide.

The present disclosure further relates to use of the microfluidic cartridge for testing viability of microorganism towards the agents.

### BRIEF DESCRIPTION OF THE ACCOMPANYING FIGURES

FIG. 1 describes a protocol illustrating the steps involved in the agent susceptibility assay, according to one embodiment.

FIG. 2 illustrates a schematic top view of the microfluidic cartridge having array of micro chamber, according to an embodiment of the present disclosure.

FIG. 3 illustrates a schematic top view of a variant of the microfluidic cartridge of FIG. 2, according to an embodiment of the present disclosure.

FIG. 4 illustrates a perspective view of a gasket used to seal inlet port and outlet port of each micro chamber of microfluidic cartridge of FIG. 2 and inlet port of each micro chamber of microfluidic cartridge of FIG. 3, according to an exemplary embodiment of the present disclosure.

FIG. 5 illustrates a sectional view of the microfluidic cartridge with gasket, according to an exemplary embodiment of the disclosure.

FIG. 6 illustrates COMSOL modelling of flow parameter inside the reaction chamber of the microfluidic cartridge.

FIG. 7 illustrates CAD design of the microfluidic cartridge.

FIG. 8 illustrates auto fluorescence of PMMA material, wherein n=3, Std deviation is <10%.

FIG. 9 illustrates concentration vs time graph at two different points in the reaction chamber of the microfluidic chamber.

FIG. 10 illustrates air-drying of reagents in the microchamber of the microfluidic cartridge and change in chip weight vs time.

FIG. 11 illustrates reagent diffusion across the microchamber of the microfluidic cartridge.

FIG. 12 illustrates diffusion of dye mixed media across reaction chamber.

FIG. 13 illustrates lamination efficiency and absence of cross contamination in the microfluidic cartridge.

FIG. 14 illustrates susceptibility of Escherichia coli ATCC 25922 QC strain to Amikacin in the microfluidic cartridge.

FIG. 15 pictorial representation of susceptibility of E. coli ATCC 25922 QC strain against Amikacin through change in the colour of the indicator dye.

FIG. 16 illustrates that auto-fluorescence of assay components at desired emission wavelengths is significantly lower when compared to fluorescence intensity of fluorophore labeled sensors (DNA based pH sensor).

FIG. 17 illustrates E. coli AST against Piperacillin within the microfluidic cartridge.

FIG. 18 illustrates the microscopic images of E. coli AST against piperacillin in the microfluidic cartridge.

FIG. 19 illustrates E.coli AST against Ceftazidime within the microfluidic cartridge.

FIG. 20 illustrates E.coli AST against Ciprofloxacin within the microfluidic cartridge.

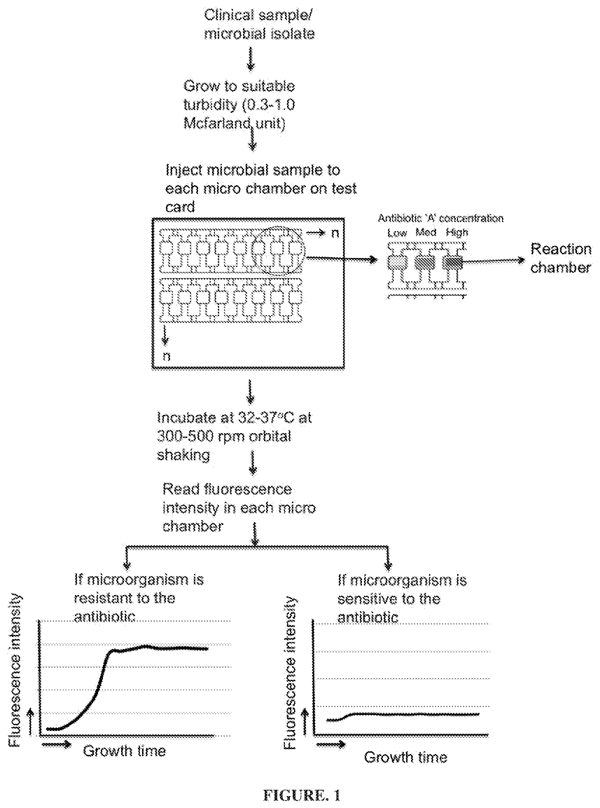

### DETAILED DESCRIPTION OF THE DISCLOSURE

The present disclosure relates to microfluidic cartridge for determining susceptibility or resistance of microorganisms towards components selected from a group comprising antibiotic, antifungal, antiviral, pesticide and insecticide.

In an embodiment of the present disclosure, the microfluidic cartridge comprises—
a non-reactive base comprising a plurality of inlaid, spatially-isolated arrays
each array comprising at least one reaction chamber wherein said reaction chamber is in fluid communication with an inlet port and an outlet port opposed to the inlet port, said base having at least one microchannel in fluid communication with all the inlet ports,
at least one separate microchannel in fluid communication with all outlet ports
and a laminar film disposed over the arrays and reaction chambers so as to sealably isolate their contents from the external environment.

In an embodiment of the present disclosure, the cartridge comprises a gasket disposed between adjacent arrays to fluidly separate the arrays.

In an embodiment of the present disclosure, the gasket is defined with demarcated points for injecting an analyte sample into each inlet port of the arrays.

In an embodiment of the present disclosure, the gasket is configured to sealably isolate adjacent arrays by means of a plurality of teeth interlocking onto groves on the bases.

In an embodiment of the present disclosure, the gasket is composed of silicone or such other biologically compatible, self-sealing material.

In an embodiment of the present disclosure, the inlet port of each array is disposed higher than its opposed outlet port. The inlet ports are longer than the outlet ports.

In an embodiment of the present disclosure, the plurality of microchannels in each of array of the plurality of arrays are spatially isolated from one another to reduce cross contamination.

In an embodiment of the present disclosure, the base substrate is laminated with a lamination film on top side.

In an embodiment of the present disclosure, the base substrate is made of a polymeric material, preferably poly (methyl methacrylate) (PMMA), polypropylene, polystyrene, or any suitable biocompatible material.

In an embodiment of the present disclosure, the microfluidic cartridge comprises sensors including but not limited to pH sensors, DNA based sensors, in the reaction chamber of each of the plurality of microchannels.

In an embodiment of the present disclosure, the reaction chamber is preloaded with a microbial culture medium, a sensor, reagent and agent against with microbial viability is to be assessed.

In an embodiment of the present disclosure, the pH sensor may be any pH sensitive DNA sensor. The sensor is conjugated to fluorophore or dye. When a change in pH is sensed by the pH sensor due to the growth of microorganisms, the sensor exhibits increased fluorescence which is read by the fluorescence reader connected to the test cartridge. When there is no microbial growth, the pH sensor does not exhibit increased fluorescence. In this way, resistance or susceptibility of the microorganisms to any agent can be determined.

In an embodiment of the present disclosure, the microfluidic cartridge comprises about 20 to 120 microchambers, wherein different or same agents having different or same concentrations, or a combination thereof can be screened. In an exemplary embodiment, the microfluidic cartridge of the present disclosure enables in vitro testing of at least 5 different agents in at least three different concentration against a clinical test sample.

In an embodiment of the present disclosure, microfluidic cartridge is designed such that there is no requirement of active mixing sample and reagents for their diffusion in the microchambers. Further, the proximity of the top and bottom walls is sufficient to ensure uniform sample distribution in the microchambers and ensures minimal bubble trapping.

In an embodiment of the present disclosure, the microfluidic cartridge provides for highly personalized in vitro assessment of resistance or susceptibility of the microorganisms to any agent, as the said microfluidic cartridge is capable of housing growth media, agent, sensor and reagents and there is only requirement of only loading the clinical sample or microbial isolate that needs to be assayed for its susceptibility or resistance to an agent. Moreover, the microfluidic cartridge is capable of assaying susceptibility or resistance of microorganisms against varying concentrations of agent in a high throughput manner.

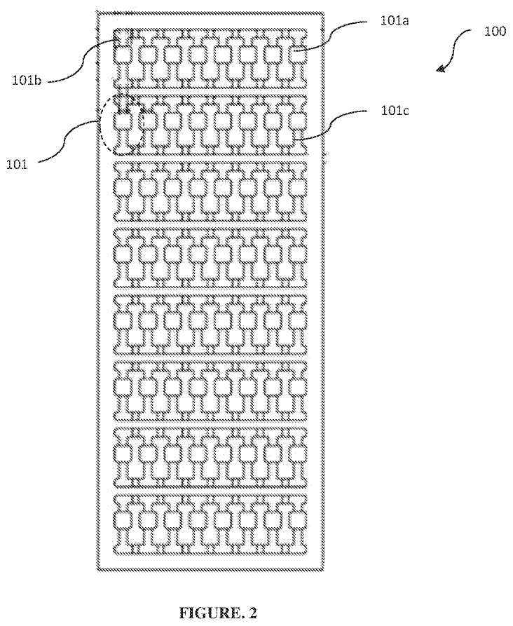

In an exemplary embodiment of the present disclosure, the FIG. 2 illustrates a schematic top view of the microfluidic cartridge. The microfluidic cartridge (100) contains a plurality of micro-chambers (101a-101n collectively referred as 101) arranged in a form of one or more arrays. Each of the plurality of micro-chambers (101), are geometrically or spatially isolated from one another to reduce cross contamination. In an embodiment of the disclosure, the microfluidic cartridge (100) may be made of a polymeric material of a defined thickness. In an embodiment of the disclosure, the plurality of micro-chambers (101) may be laminated wising opaque low background lamination film of Polypropylene (PP), Polyphenylene ether (PPE), Polymethyl methacrylate (PMMA) material or suitable material at bottom side and optically clear material laminated at top side of the microfluidic cartridge. In another embodiment of the disclosure, the bottom side of the microfluidic cartridge (100) is solid, opaque, black or any other suitable color and topside has open micro-chambers, which is laminated using optically clear film.

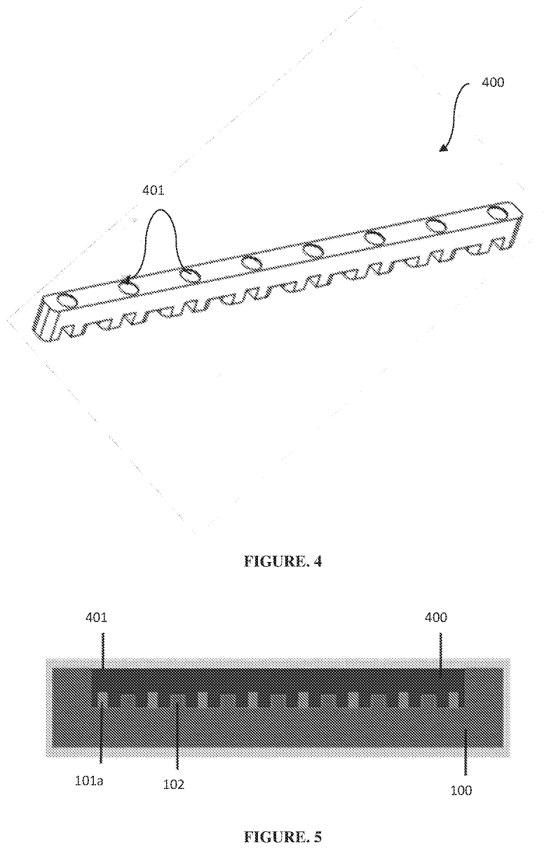

In an embodiment of the present disclosure, the microfluidic cartridge (100) may be made of a polymeric material such as but not limiting to Polymethyl methacrylate) (PMMA), and the micro-chambers (101) may be formed by micro milling, laser cutting, injection molding, vacuum casting or any other suitable process. Each micro-channel may be sealed at inlet channel (101b) using gasket (400) as shown in FIG. 4 such that each of the micro-chambers are isolated.

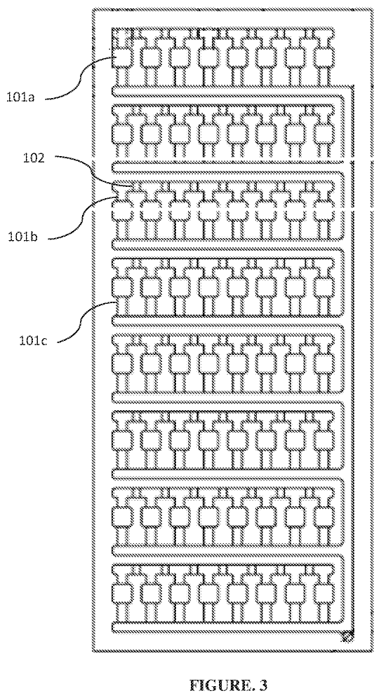

In an embodiment of the present disclosure, the microfluidic cartridge (100) may include a plurality of micro-channels (101) arranged in arrays. Each of the plurality of micro channel (101) includes a reaction chamber (101a), inlet and outlet ports (101b and 101c) at either ends of the reaction chamber (101a). The reaction chamber (101a) of each of the plurality of micro-chambers (101) may be integrated with at least one sensor including but not limiting to DNA-based pH sensor [not shown]. The reaction chamber (101a) may be preloaded with antibiotic, microbial growth media. The sample to be tested can be inserted into the reaction chamber (101a) through an inlet port (101b). In another configuration as shown in FIG. 3 the outlet port (101c) of each reaction chamber (101a) is connected to a secondary channel for each row that further connected to primary channel that opens outside of the microfluidic cartridge (100).

In an embodiment of the present disclosure, the micro fluidic cartridge (100) includes a plurality of gasket tooth sealing positions, called separators (102) to give stability for gasket sealing and seal entry and exit ports (101b and 101c) of the reaction-chamber (101a), which allows sample loading and sterility of the reaction chamber (101a) environment. In an embodiment of the present disclosure, each of the plurality of gaskets (400) [FIG. 4] may be made of silicon or suitable polymer material. In an exemplary configuration, as shown in FIG. 5, the gasket puncture site (401) at channel entry has at least 300-700-μm space to load the sample, and there are separators (102) of 300-600 μm that isolate each micro channel (101) in FIG. 2 and inlet port (101b) and reaction chamber (101a) in FIG. 3. The gasket shown in FIG. 4 seals on the top of each separator (102) and avoid cross-contamination.

The present disclosure further relates to a kit comprising the microfluidic cartridge containing growth media, agent, sensor and reagent and an instruction manual, providing instructions to use the said microfluidic cartridge for determining the susceptibility or resistance of microorganisms towards the agent.

In an embodiment of the present disclosure, the kit provides for highly personalized in vitro assessment of resistance or susceptibility of the microorganisms to any agent. The kit also provides for in vitro assessment of susceptibility or resistance of microorganism against varying concentration of agent in a high throughput manner.

In an embodiment of the present disclosure, the method of assembling the kit comprises—
loading growth media, agent, sensor and reagents, consecutively in any order or sequence into the microfluidic cartridge; or
providing growth media, agent, sensor and reagent, respectively in separate containers along with the microfluidic cartridge; and
providing instruction manual having details regarding the growth media, agent, sensor and reagent and instruction as to how to carry out in vitro assessment of susceptibility or resistance of microorganism towards the agent.

In an embodiment of the present disclosure, the kit enables in vitro assessment of susceptibility or resistance of microorganism towards agent in about 8 hours, preferably in about 5 hours. Thus, the kit enables rapid in vitro assessment of susceptibility or resistance of microorganism against any agent.

The present disclosure further relates to an assay, particularly in vitro assay for determining resistance or susceptibility of microorganisms towards agent by employing the microfluidic cartridge or the kit described above.

In an embodiment of the present disclosure, the assay detects susceptibility or resistance of microorganism towards antibiotics by using pH change as an indicator with the help of sensors in the microfluidic cartridge or the kit described above.

In another embodiment of the present disclosure, the sample comprising microorganisms or the microbial isolate to be tested is added to the microfluidic cartridge in an appropriate manner comprising growth media, agent, sensor and reagent. The growth of the microorganism in the sample or that of the microbial isolate is determined by the change in the pH of the growth media. If the microorganism present in the clinical sample or that of the microbial isolate is susceptible to the agent, then there would be no growth of the microorganism, as a result of which the pH would remain constant. However, when the microorganism is resistant to the agent, the microorganism in the clinical sample or that of the microbial isolate would grow in the presence of the growth media, as a result of which the pH would change. This change in pH is detected by the pH sensor which exhibits an increased fluorescence intensity, thus confirming resistance of the microorganism to the agent.

In an embodiment of the present disclosure, the sensor is a pH sensitive DNA based sensor.

In an exemplary embodiment of the present disclosure, the test sample may be any sample, such as urine, positive blood cultures, ascitic fluid, peritoneal fluid and explant, soil, and any agriculture sample. In yet another embodiment of the present disclosure, the sample may be a microbial isolate.

In an embodiment of the present disclosure, the assay may be performed for determining susceptibility or resistance of microorganism, including but not limited to bacteria, yeast, and fungi.

In an embodiment of the present disclosure, the growth media may be any microbial growth media supporting the growth of the microorganism.

In an exemplary embodiment of the present disclosure, the steps involved in performing the assay for determining susceptibility or resistance of microorganism towards agent comprises:
(a) obtaining a clinical sample/microbial isolate;
(b) growing the microorganism in the sample or isolate to a suitable turbidity;
(c) introducing the microorganism of step (b) into each microchamber of the microfluidic cartridge containing growth media, agent, pH sensor and reagents;
(d) incubating the microfluidic cartridge at a predetermined temperature with or without shaking; and
(e) reading fluorescence intensity in each microchamber of the microfluidic cartridge to determine the susceptibility or resistance of the microorganism towards the agent.

In an embodiment of the present disclosure, each microchamber of the microfluidic cartridge comprises different agent at same concentration or varying concentration or different concentrations of the same agent.

The results of the assay in the present disclosure are obtained within about 8 hours, more preferably within about 5 hours and is thus a rapid method, extremely beneficial in clinical applications.

In an embodiment of the present disclosure, the assay of the present disclosure may also be used for determining response of the microorganisms towards insecticide pesticides or herbicides.

In another embodiment, the present disclosure relates to a method for assessing microbial viability of an analyte sample against an agent comprising steps of:
Inserting analyte sample through the demarcated portions of the gasket into the preloaded reaction chambers of the cartridge;
Incubating the cartridge at predetermined temperature and duration with constant shaking;
Obtaining a read-out from each reaction chamber of the cartridge; and
Determining the viability of the microbe.

In an embodiment of the present disclosure, the analyte sample is selected from a group comprising serum, ascites, sputum, blood, other body-fluids and culture isolates.

In an embodiment of the present disclosure, the viability is determined concurrently at different concentrations or dosages of the agent.

In an embodiment of the present disclosure, viability can be determined against a plurality of agents simultaneously.

In an embodiment of the present disclosure, the viability of microbe is based on intensity observed through photometry, fluorescence, luminescence or colorimetry.

The present disclosure also relates to use of the microfluidic cartridge in screening agents to assess their response against microorganisms, wherein the varying concentrations of same or different agents or same concentration of same or different agents are assessed for their ability to kill the microorganisms, particularly pathogens.

In an embodiment of the present disclosure, the advantages noted by the present disclosure is outlined below—
1. The microfluidic cartridge of the present disclosure has an array of about 20 to 120 microchambers that suitably house components, such as growth media, agent, pesticide, herbicide, pH sensor, reagents to perform in vitro assay. Thus, the cartridge enables screening multiple agent, pesticide or herbicide at varying concentrations or at same concentration.
2. The microfluidic cartridge of the present disclosure comprises a safe seal that allows loading of sample comprising microorganism or microbial isolate into each microchamber. It keeps microchambers isolated from outside environment or contamination from the air.
3. The assay of the present disclosure utilizing microfluidic cartridge is a tool in rapid and personalized agent screening.
4. The assay of the present disclosure utilizing microfluidic cartridge may be used in companion diagnostics to test response of a patient towards antibiotic regime in critical care units or elsewhere.
5. The assay of the present disclosure utilizing microfluidic cartridge may be used in screening of microbial response for agricultural application such as microbial response to pesticides, insecticide or herbicides.
6. The assay of the present disclosure utilizing microfluidic cartridge may be used in screening of susceptibility or resistance of microorganisms in veterinary applications.

As used in the instant specification, microbial viability means susceptibility or resistance of the microorganism towards agent selected from a group comprising antibiotic, antifungal antiviral pesticide, herbicide and insecticide.

Additional embodiments and features of the present disclosure will be apparent to one of ordinary skill in art based upon the description provided. The embodiments provide various features and advantageous details thereof in the description. Descriptions of well-known/conventional methods and techniques are omitted so as to not unnecessarily obscure the embodiments. The example provided herein is intended merely to facilitate an understanding of ways in which the embodiments provided may be practiced and to further enable those of skilled in the art to practice the embodiments provided. Accordingly, the following example should not be construed as limiting the scope of the embodiments.

### EXAMPLE 1: ASSAY TO DETERMINE THE SUSCEPTIBILITY OR RESISTANCE OF MICROORGANISM USING THE MICROFLUIDIC CARTRIDGE

The agent susceptibility assay was carried out in a microfluidic cartridge. This assay was also carried out in a micro-titer plate as a standard reference control. Bacterial/clinical sample grown at suitable turbidity or at least about 0.3 to about 1.0 McFarland unit was transferred to the microchamber. The microchamber had microbial growth sensors (pH sensor) at concentration of about 5 μM to about 50 μM, cation adjusted Muller Hinton broth or suitable microbial growth media, and antibiotics concentration based on CLSI guidelines. The total reaction volume range was from about 6 μl to about 150 μl. Plate/test card were sealed using plate sealer or lamination film and incubated at about 32° C. to about 37° C., at about 300-500 rpm orbital shaking. The change in the fluorescence intensity of pH sensors was measured using fluorescence reader after every 20-60 minutes till about 4-7 hours incubation period. Thus, the susceptibility or resistance of the microorganism was assessed towards the agent.

### EXAMPLE 2: DESIGN AND COMSOL MODELING OF MICROFLUIDIC CARTRIDGE

COMSOL modelling of microchamber and flow in the microchamber was analyzed. In the laminar flow simulation, stationary phase study was performed using sample fluid. Density and dynamic viscosity of water at room temperature was 997 Kg/m³ and 8.91e⁻¹ Kg/(m·s) respectively. Boundary conditions for all the wall were no slip boundary condition. Inlet velocity conditions are normal inflow velocity of magnitude 10 μm/s coupled with pressure point constraint P=1 atm. Time dependent study for transport of dilute species coupled with laminar flow was performed on 5 mol/m³ concentrated glucose. Diffusion coefficient of glucose at room temperature is 6.8e-9m²/s. No flux boundary condition is used for all the boundaries. Glucose was used in place of dried media in the reaction chamber, dried layer of glucose was considered as 250 μm thick.

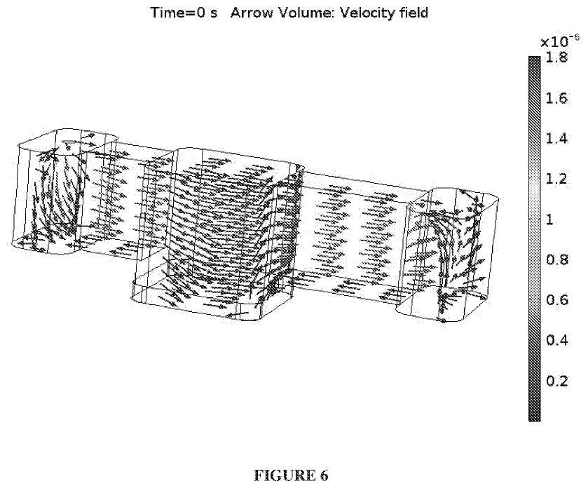

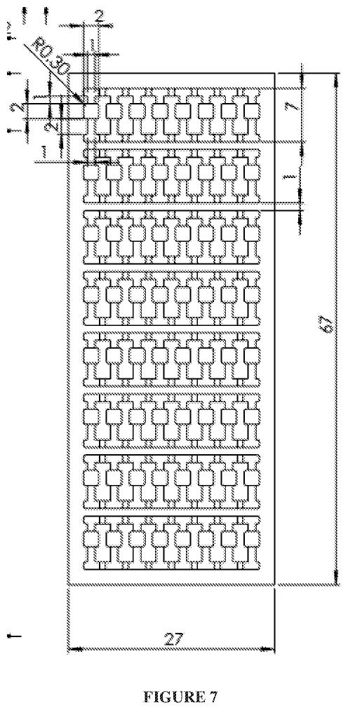

Flow was observed to be laminar through the chamber with higher velocity of 1.2-1.8×10⁻⁶ at inlet port and no turbulence at chamfered edges (illustrated in FIG. 6). Due to low Reynolds number no counter flow was observed at chamfered edge which will reduces chances of cavitation and trapping air in between. This will ensure uniform media in the microchamber for phenotypic assays and readout.

Microfluidic chip had about 64 microchambers each with maximum volume capacity of 8 μl. chip dimension was 27×67×3 mm (W×L×H) containing an array of 8 microchambers in 8 rows (illustrated in FIG. 2). All microchambers in one row had one channel connecting entry port however, each microchamber was isolated using extruded barrier between the chambers to be sealed later using silicon gasket and lamination on the top. Barrier between the microchamber(s) ensures that there is no cross-contamination between the chambers.

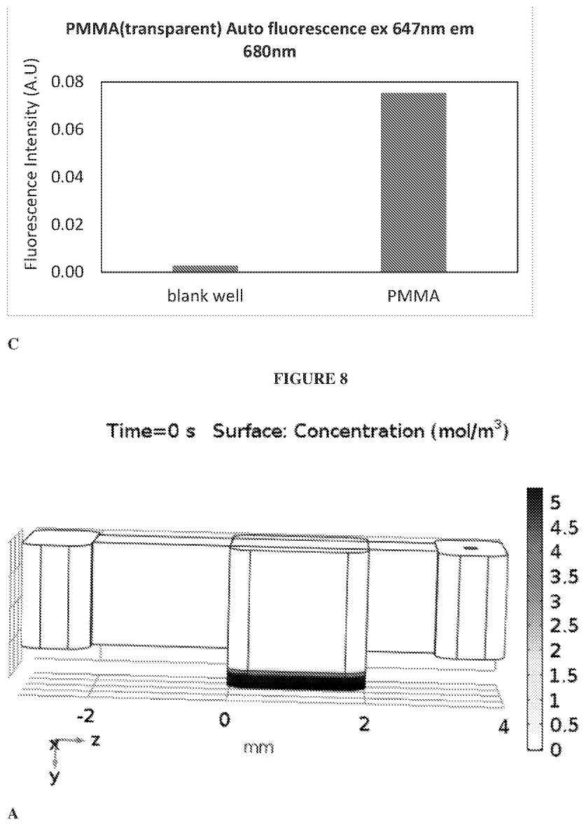

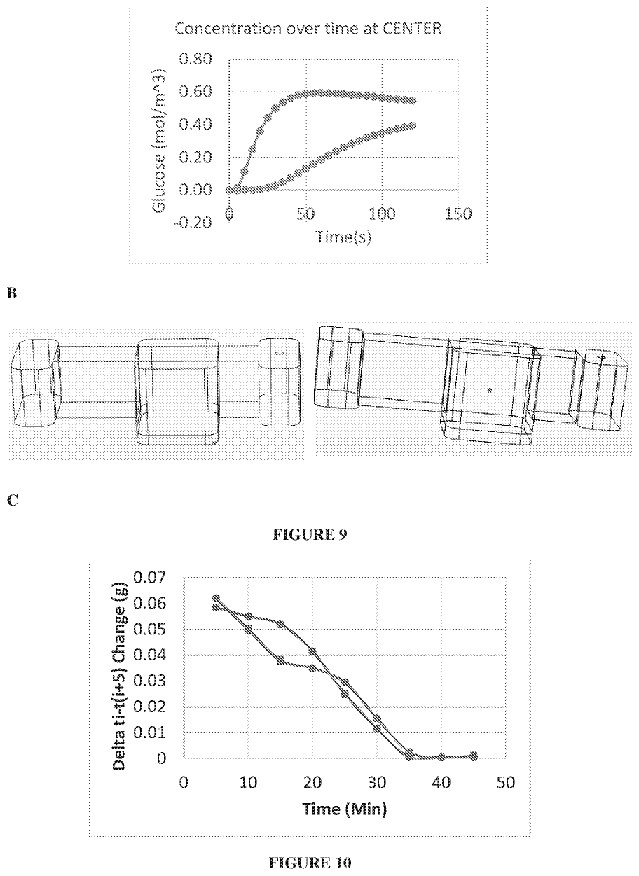

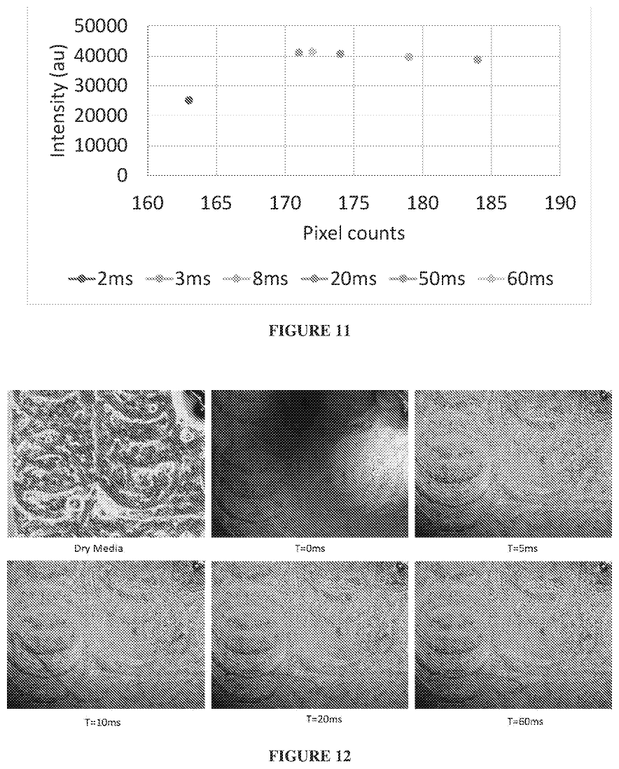

The flow parameters and reagent diffusion are important to achieve uniform concentration of assay reagents in the microchamber. Time dependent modeling of flow coupled with mass was simulated using COMSOL. Simulation condition used were at room temperature, inlet of the sample is controlled by initial velocity of 5 mm/s and point pressure outlet condition. Glucose is used as model material having concentration about 5 mol/m³ with diffusivity 6.8×10⁻⁹ at room temperature. The concentration of glucose dropped down to 1.1 mol/m³ at the bottom of the reaction chamber within 60 seconds after injecting water, which indicates that dried media has started diffusing and within 2 minutes concentration of glucose across the chamber was 0.5 mol/m³. It also indicates that the concentration of glucose is evenly distributed throughout the chamber (illustrated in FIG. 9).

Diffusion modelling data was validated experimentally. Cartridge was sterilized under UV light for about 20 minutes. About 4 μl of reagent was added in each microchamber and air dried at 37° C. reagents contained cation adjusted Muller Hinton broth with 0.05% phenol red as sensor. Weight of the cartridge was measured at about 5 minutes interval till no change in cartridge weight was recorded. At 37° C. incubator, there was no change in weight was observed after 35 minutes. Assay in the chamber was completely dried after about 35 minutes. The trend of drying was linear, after the said drying time it became constant. Optimal air-drying condition is drying at temperature of about 37° C. for about 40 minutes.

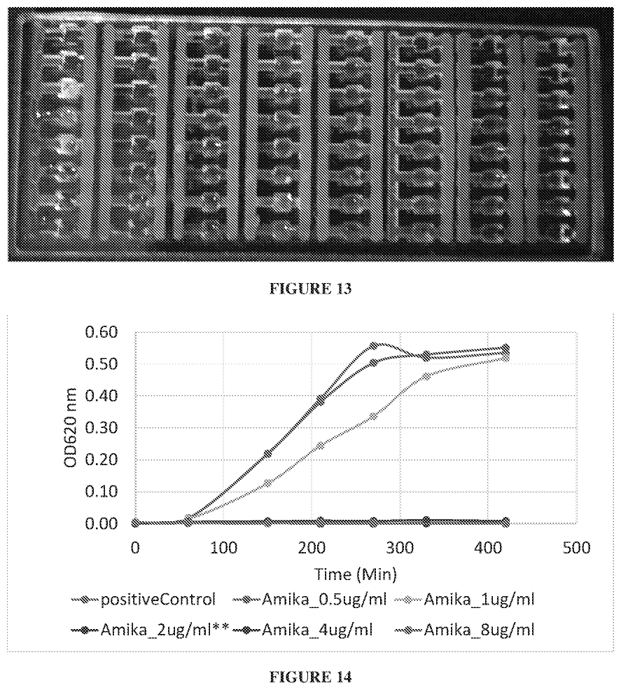

In order to maintain standard (aspartate aminotransferase) AST test conditions there should not be any cross contamination in the microfluidic cartridge. The efficiency of lamination and isolation of microchamber (no-cross contamination) was tested. The cartridge was cleaned with water and then rinsed with 70% ethanol. The cartridge was exposed to UV light inside the laminar hood for about 30 minutes. The cartridge was sealed with PCR lamination sheet. For this experiment cation adjusted muller broth was injected into the microchamber using syringe, under sterile condition. The cartridge was incubated at room temperature for about 24 hours to test the stability and efficiency of lamination. No cross contamination was observed at the end of the experiment (illustrated in FIG. 13).

### EXAMPLE 3: MICRO-FABRICATION OF MICROFLUIDIC CARTRIDGE, CARTRIDGE ACCESSORIES AND LAMINATION

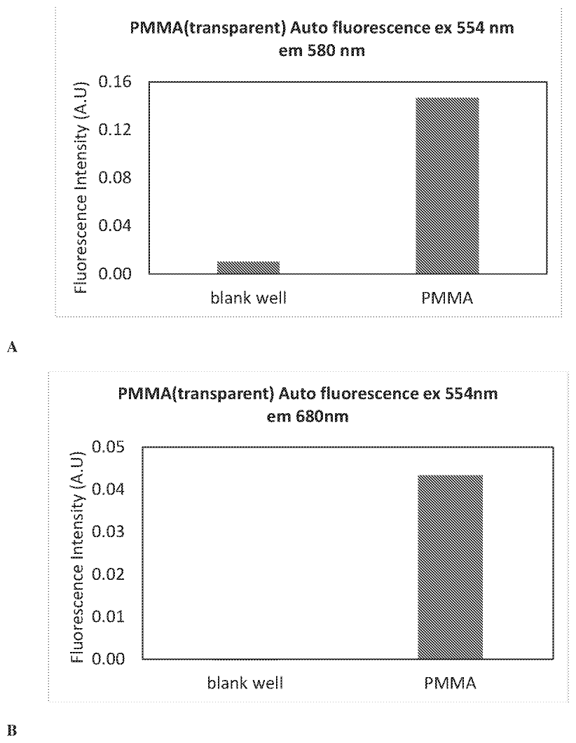

Auto florescence of different grade of poly (methyl methacrylate) (PMMA) sheets were tested to assay to select microfluidic cartridge material with least auto fluorescence. PMMA sheet was cut into small circle, such that it can fit into the wall of 96 black well plate and auto fluorescence was measured for excitation at 554 nm emission at 580 nm and 680 nm and excitation at 640 nm emission at 680 nm. PMMA sample was measure with the reference of blank well on the plate (illustrated in FIG. 8)

PMMA sheets have slightly more background with respect to the empty black wells. However, with respect to sensors, the background signal of PMMA is negligible as fluorescence intensity of sensors in our assay is in between 2.7 au to 27 au, which is significantly larger than PMMA background signal.

### EXAMPLE 4: OPTIMIZATION OF CULTURE AND SENSOR CONDITIONS ON MICROFLUIDIC CARTRIDGE

Agent susceptibility was tested on microfluidic cartridge and the outcome compared with optimized 96 well plate. Phenotypic assay was optimized on 96 well pate, briefly, an overnight grown E. coli ATCC25922 culture was dispersed in cation adjusted muller Hinton broth to prepare inoculum of 0.5 MCF unit. For this experiment 0.5 μg/ml, 1 ug/ml, 2 μg/ml and 4 μg/ml amikacin dilution was prepared by diluting stock solution of 1.6 μg/ml in cation adjusted muller Hinton broth E. coli ATCC 25922 showed susceptibility at 2 μg/ml amikacin concentration (illustrated in FIG. 14). according to CLSI guidelines

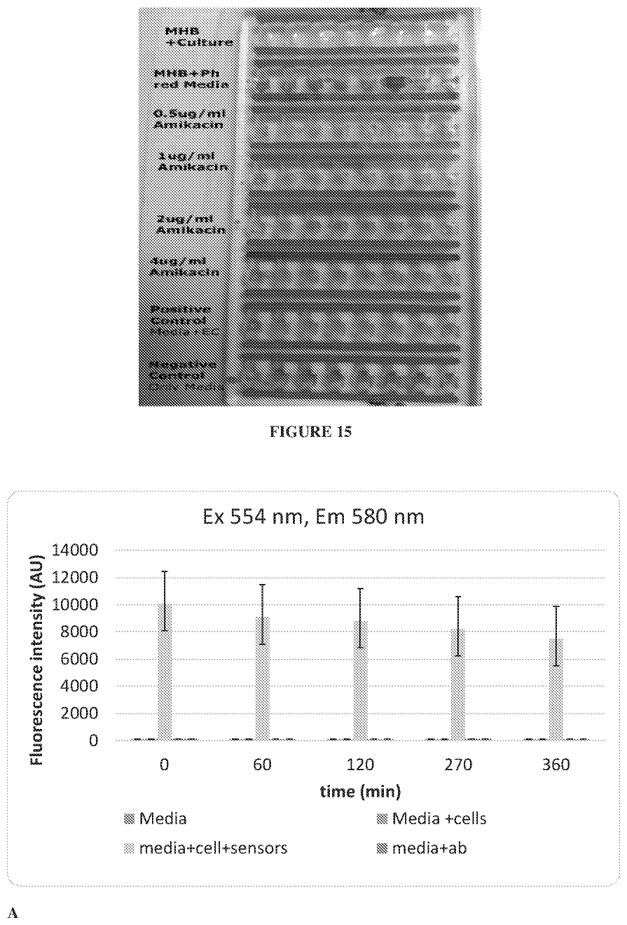

Qualitative test was performed to check microbial growth in the microfluidic cartridge. Phenol red (about 0.05% w/v) containing cation adjusted Muller Hinton broth was used as susceptibility indicator. Microbial growth changes pH and Phenol red changes color with pH variation. Thus, a colour change could provide a read-out on the susceptibility or resistance of a microorganism. No color change would indicate no growth as a response to antibiotics. Briefly, microfluidic cartridge was cleaned with water and then rinsed with 70% ethanol and then UV sterilized by exposing under UV light for about 30 minutes. To compare the susceptibility test preformed on the microfluidic cartridge and 96 well plate, master mix of about 150 μl was prepared on 96 well plate and 6 μl o the master mix was used in microchamber of the microfluidic cartridge. Both 96 well plate and microfluidic cartridge was sealed with lamination film and incubated at 37° C. and change in color intensity was recorded (illustrated in FIG. 15). The FIG. 15 depicts change in the color of indicator dye at 0.5 and 1 ug/ml amikacin concentration and no change in the color intensity for concentration above 2 μg/ml. The results were obtained in 3 hour and design of the microfluidic cartridge was suitable for microbial growth and no cross contamination was observed.

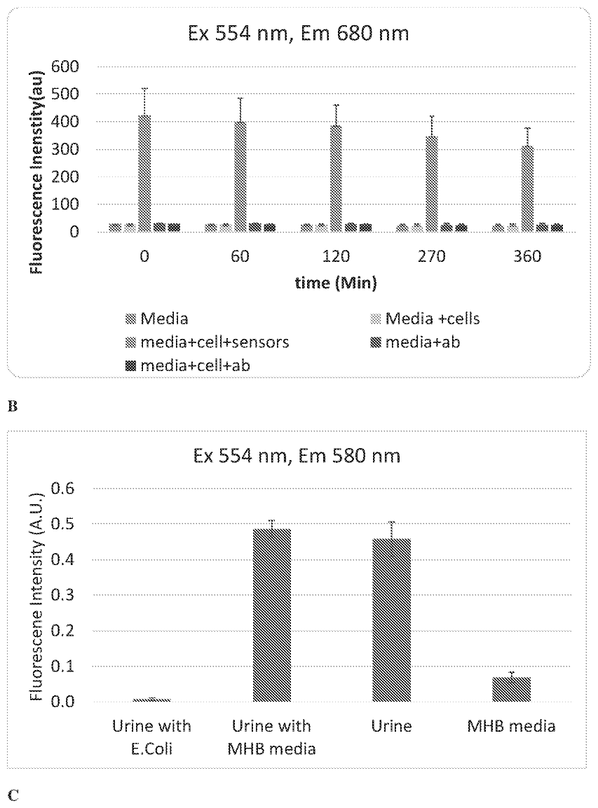

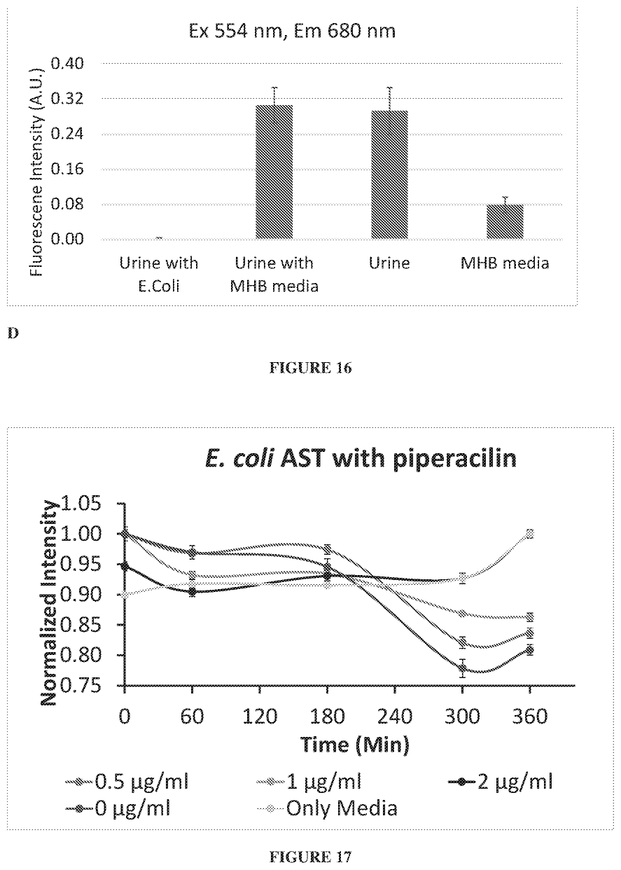

The susceptibility assay is based on Förster Resonance Energy Transfer (FRET) mechanism. DNA sensors are conjugated with a donor and acceptor fluorophore. Culture media and the sample may interfere in fluorescence assay. The autofluorescence of assay components such as CAMHB, antibiotics, urine sample and bacterial culture were tested at 554 nm excitation, 584 nm and 680 nm emissions (illustrated in FIG. 16). The FIG. 16 depicts that the auto fluorescence of assay components at said emission wavelengths was significantly lower when compared to fluorescence intensity of fluorophore labelled DNA sensors.

### EXAMPLE 5: AGENT SUSCEPTIBILITY TEST USING MICROFLUIDIC CARTRIDGE

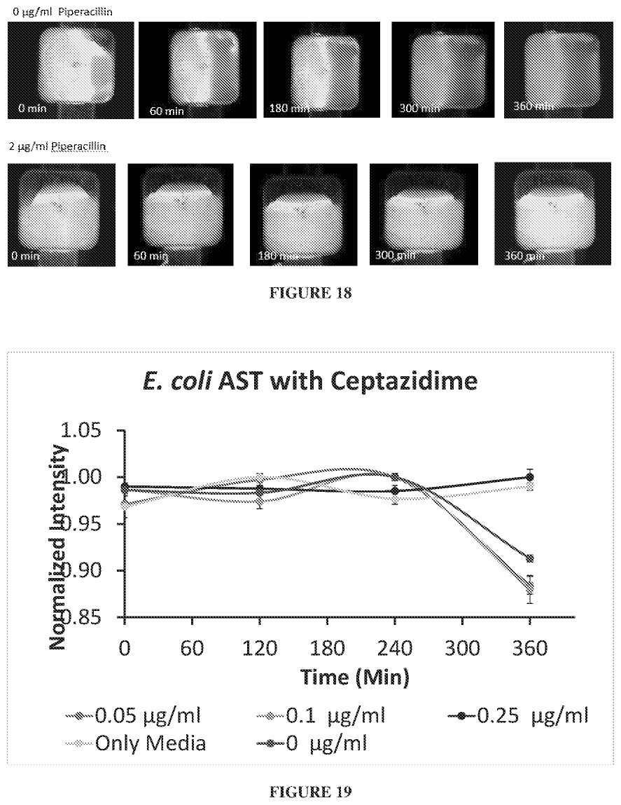

About 5 μM sensor was prepared and stored at about 4° C. overnight for equilibration of the sensors. Overnight grown cultures of E. coli, S. aureus, E. faecalis was picked from LB agar plate and dispersed in cation adjusted Mueller Hinton broth with 1% glucose. The concentration of the cells were adjusted to 0.5 MFU. antibiotic dilution was prepared according to CSI guidelines. Stock solutions were prepared in 96 well plate using 23 μl of cation adjusted Muller Hinton broth, 25 μl of desired concentration of antibiotic and 2 μl of 5 μM sensors. Then 50 μl of 1×10⁶ CFU/ml inoculum was added in wells to achieve inoculum size 5×10⁵ CFU/ml in final assay volume. Each stock solution was loaded on microfluidic cartridge. Microfluidic cartridge was sealed with polypropylene sealing film and incubated at 37° C. with 70% humidity. The change in the donor fluorescence intensity was recorded till 6 hour incubation. Each microchamber of the microfluidic cartridge was image using 1×73 Olympus inverted fluorescence microscope. Images were analyzed using ImageJ version 1.8.0. the average was taken over replicate and standard deviation was calculated (illustrated in FIG. 18).

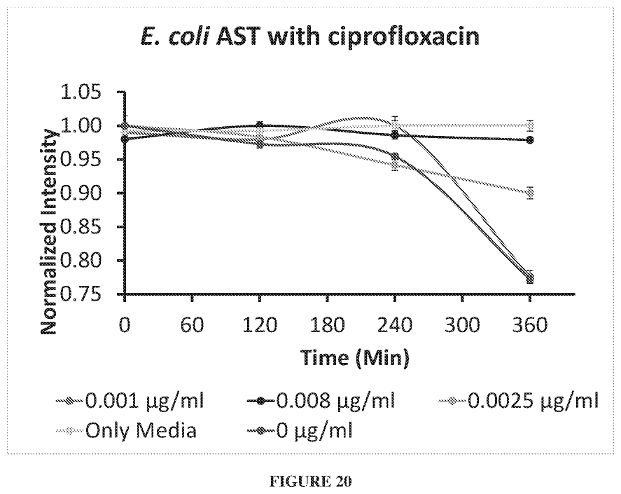

The FIGS. 17, 19 and 20, respectively depicts the susceptibility of E. coli against piperacillin, ceftazidime, and ciprofloxacin. E. coli showed sensitivity cut of value of about 2 μg/ml, about (125 μg/ml and about 0.008 μpg/ml for piperacillin, ceftazidime and ciprofloxacin, respectively. No change in fluorescence intensity was recorded in microchambers that contained only media (media+sensor). Other microchambers had growth media, sensors, microorganism and antibiotics. Microorganisms were grown in the media at lower concentration of antibiotics, changing pH of the medium which in turn changes the structure of the sensor to create FRET, reporting reduced donor fluorescence intensity over an incubation time.

Table of referral numerals

| Reference number | Description |
|---|---|
| 100 | Microfluidic cartridge |
| 101 | Micro-chambers |
| 101a | Reaction chamber |
| 101b | Inlet port |
| 101c | Outlet port |
| 102 | Separator |
| 400 | Gasket |
| 401 | Gasket puncture site |

1. A microfluidic cartridge for assaying microbial viability to an agent comprising:
a non-reactive base comprising a plurality of inlaid, spatially-isolated arrays
each array comprising at least one reaction chamber wherein said reaction chamber is in fluid communication with an inlet port and an outlet port opposed to the inlet port,
said base having at least one microchannel in fluid communication with all the inlet ports,
at least one separate microchannel in fluid communication with all outlet ports and a laminar film disposed over the arrays and reaction chambers so as to sealably isolate their contents from the external environment.

2. The cartridge according to claim 1 comprising a gasket disposed between adjacent arrays to fluidly separate the arrays.

3. The cartridge according to claim 2, wherein the gasket is defined with demarcated points for injecting an analyte sample into each inlet port of the arrays.

4. The cartridge according to claim 2, wherein the gasket is configured to sealably isolate adjacent arrays by means of a plurality of teeth interlocking on to grooves on the base.

5. The microfluidic cartridge according to claim 2, wherein the gasket is composed of silicone or such other biologically compatible, self-sealing material.

6. The microfluidic cartridge according to claim 1, wherein the inlet port of each array is disposed higher than its opposed outlet port.

7. The microfluidic cartridge according to claim 1, wherein inlet ports are longer than the outlet ports.

8. The microfluidic cartridge according to claim 1, wherein the base is composed of non-reactive material selected from the group comprising of PIVINIA, polypropylene, polystyrene and such other suitably moldable material.

9. The microfluidic cartridge according to claim 1, wherein the reaction chamber is preloaded with a microbial culture medium, a sensor, reagent and agent against which microbial viability is to be assessed, wherein the sensor is pH sensor, DNA based pH sensor, and wherein the reagent is fluorophore.

10. The microfluidic cartridge according to claim 1, wherein the cartridge is optically opaque excepting a transparent window above each reaction chamber.

11. The microfluidic cartridge according to claim 9, wherein the preloaded agent is an antibiotic, antiviral, insecticide or pesticidal composition.

12. A method for assessing microbial viability of an analyte sample against an agent comprising the steps of:
a) Inserting analyte sample through the demarcated portions of the gasket into the preloaded reaction chambers of the cartridge according to claim 9;
b) Incubating the cartridge of (a) at predetermined temperature and duration with constant shaking;
c) Obtaining a read-out from each reaction chamber of the cartridge (b); and
d) Determining the viability of the microbe.

13. The method according to claim 12, wherein the analyte sample is selected from a group comprising serum, ascites, sputum, blood, other body-fluids and culture isolates.

14. The method according to claim 12, wherein viability is determined concurrently at different concentrations or dosages of the agent.

15. The method according to claim 12, wherein viability can be determined against a plurality of agents simultaneously.

16. The method according to claim 12, wherein the viability of microbe is based on intensity observed through fluorescence, luminescence or colorimetry.

17. A kit comprising the microfluidic cartridge according to claim 1 housing growth media, agent, sensor and reagent and instruction manual having instructions to determine susceptibility or resistance of microorganisms towards the agent.

18. The kit according to claim 17, wherein the growth media is selected from a group comprising Muellar hinton broth, cation adjusted Muellar hinton broth, Muellar hinton supplemented with sheep or horse blood; wherein the agent is selected from a group comprising antibacterial, antifungal, antiviral, pesticide and insecticide; and wherein the sensor is pH sensor or DNA based pH sensor.

19. Use of the microfluidic cartridge according to claim 1 for testing viability of microorganisms towards agents.
